(19) United States
(12) Patent Application Publication
(10) Pub. No.: US 2008/0149116 A1
(43) Pub. Date: Jun. 26, 2008

(54) APPARATUS FOR LEG ABDUCTION

(75) Inventor: Elizabeth Ann Ciccantelli, Albuquerque, NM (US)

Correspondence Address: VOLPE AND KOENIG, P.C., UNITED PLAZA, SUITE 1600, 30 SOUTH 17TH STREET, PHILADELPHIA, PA, 19103, US

(73) Assignee: LEG-ABDUCT LLC, Pennsauken, NJ (US)

(21) Appl. No.: 11/775,550

(22) Filed: Jul. 10, 2007

Related U.S. Application Data
(60) Provisional application No. 60/876,870, filed on Dec. 22, 2006.

(51) Int. Cl. A61F 5/37; F16B 7/10; F16C 11/00

(52) U.S. Cl. 128/882; 403/109.1; 403/113

(57) ABSTRACT

An apparatus for abducting a patient's legs is disclosed. In a preferred embodiment, the apparatus is comprised of two cuffs connected by a telescoping rod that is adjustable and lockable. The apparatus is used by a caregiver to tend to the personal needs of a disabled patient. The apparatus is positioned between the patient's legs, and the telescoping rod is locked thereby providing a fixed amount of abduction. In an alternative embodiment, a fixed length, preferably disposable support arms is used in conjunction with the cuffs for providing a low cost solution for leg abduction. Preferably, an articulating joint allows the upper cuff to adjust to a patient's body type improving comfort. Also, a flange on the base cuff improves stability of the apparatus allowing for hands-free operation.

### CROSS REFERENCE TO RELATED APPLICATION

This application claims the benefit of U.S. Provisional Application No. 60/876,870 filed Dec. 22, 2006, which is incorporated by reference as if fully set forth.

### FIELD OF INVENTION

The present invention generally relates to medical devices. More particularly, the present invention relates to an apparatus for abducting a patient's legs to access the patient's perineal area.

### BACKGROUND

Patients suffering from various debilitative disorders require intensive medical care. A patient whose debilitation has progressed to where the patient is confined to his or her bed requires constant medical attention. It is critically important for a bed confined patient to receive appropriate personal care, including general cleansing, catheterization, and various other medical procedures. However, it is often difficult for a medical practitioner to gain access to a debilitated patient's perineal area to provide care. It is especially difficult for a single practitioner to abduct a patient's legs to gain access to the perineal area. Furthermore, once the patient's legs are abducted, the patient may become restless and move slightly such that access becomes obstructed or is blocked altogether.

Accordingly, it is desirable to provide a lightweight, portable, easy-to-operate device that is comfortable for the patient and provides appropriate leg abduction.

### SUMMARY

An apparatus for abducting a patient's legs is disclosed. In a preferred embodiment, the apparatus is comprised of two cuffs connected by a telescoping rod that is adjustable and lockable. The apparatus is used by a caregiver to tend to the personal needs of a disabled patient. The apparatus is positioned between the patient's legs, and the telescoping rod is locked thereby providing a fixed amount of abduction. In an alternative embodiment, a fixed length, preferably disposable support arms is used in conjunction with the cuffs for providing a low cost solution for leg abduction. Preferably, an articulating joint allows the upper cuff to adjust to a patient's body type improving comfort. Also, a flange on the base cuff improves stability of the apparatus allowing for hands-free operation.

### BRIEF DESCRIPTION OF THE DRAWINGS

The foregoing summary, as well as the following detailed description of the preferred embodiments of the invention will be better understood when read in conjunction with the appended drawings. For the purpose of illustrating the invention, there are shown in the drawings embodiments which are presently preferred. It should be understood, however, that the invention is not limited to the precise arrangement shown.

FIG. 1 is a perspective view of an apparatus for leg abduction in accordance with a preferred embodiment of the present invention.

FIG. 2 is an exploded view of the base portion of the apparatus as shown in FIG. 1.

FIG. 3 is a perspective view of the base assembly of the apparatus of FIG. 1.

FIG. 4 is an exploded view of the upper assembly of the apparatus of FIG. 1.

FIG. 5 is a perspective view of the upper assembly interconnected with the base assembly.

FIGS. 6A and 6B shows the articulating motion of the cuff located on the upper assembly in accordance with a preferred embodiment of the present invention.

FIG. 7 is an exploded view of the interconnection of the upper assembly and the base assembly of the apparatus of FIG. 1.

FIG. 8 shows the apparatus of FIGS. 1 and 6 abducting the legs of a patient.

FIG. 9 is a perspective view of an apparatus for leg abduction wherein fixed length support arms are used in accordance with an alternative embodiment of the present invention.

FIG. 10 is a perspective view of the apparatus of FIG. 9 including an articulating joint on the upper cuff in accordance with a preferred embodiment of the present invention.

### DETAILED DESCRIPTION OF THE PREFERRED EMBODIMENTS

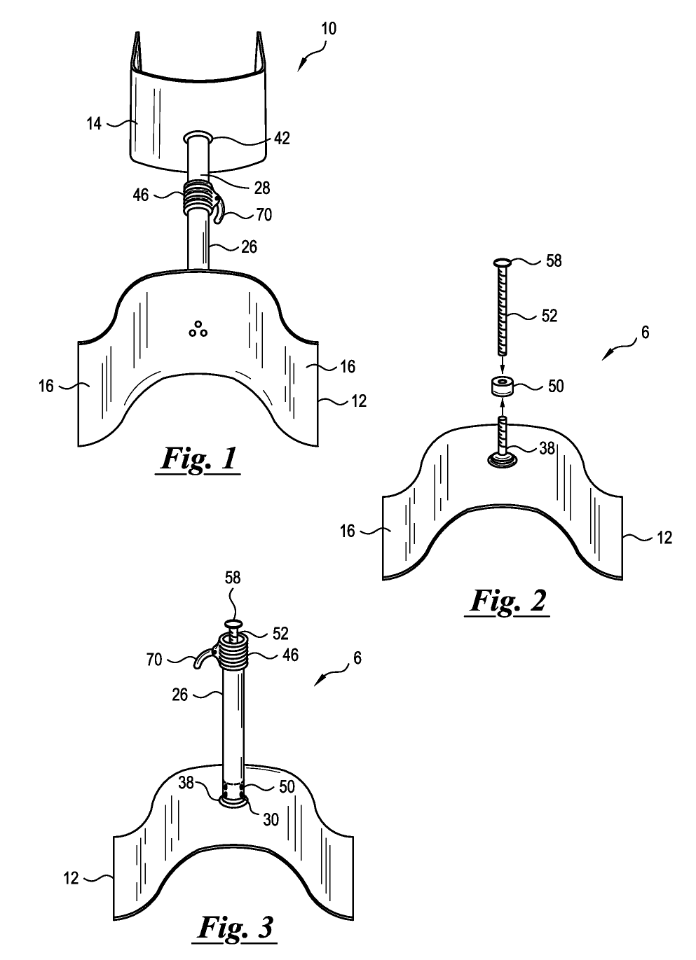

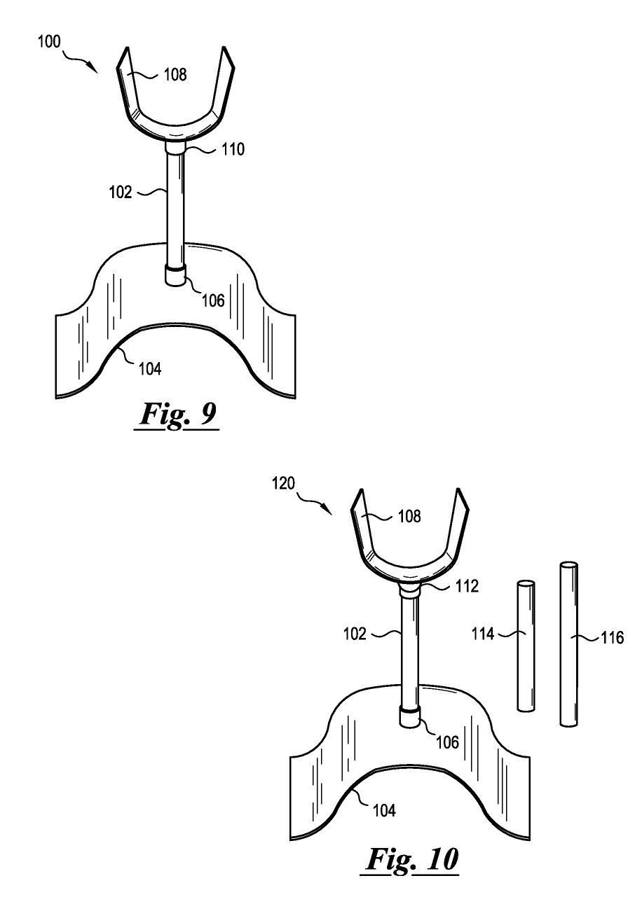

FIG. 1 shows a preferred embodiment of the apparatus for leg abduction 10 according to the present invention. The apparatus 10 has a base cuff 12 and an upper cuff 14. Preferably, the cuffs 12, 14 are comprised of a rigid material that is easily cleaned, such as a lightweight, anodized aluminum, a durable acrylic or stainless steel to allow for autoclaving. The cuffs 12, 14 may also be plastic or a semi-flexible material. The cuffs 12, 14 are shown as generally concave and curved away from each other so that a patient's legs can fit securely within (as shown in FIG. 9). The cuffs 12, 14 may formed into various shapes, such as flat or convex and may be different sizes and shapes to accommodate various patients.

Preferably, the base cuff 12 is larger than the upper cuff 14 and the base cuff 12 has a more aggressive curvature to facilitate use of the apparatus while the patient is in a side lying position, which is shown in FIG. 9. Depending on a patient's body type and size, various size and shaped cuffs may be used in combination to provide greater comfort and support. The base cuff 12 further has a flange portion 16 that provides lateral stability and enables the apparatus to be freestanding. The flange 16 frees the caregiver from balancing the apparatus and allows hands-free operation. The flange portion 16 may be substantially the same width as the cuff, or it may flare outward to provide more stability to the apparatus 10.

The cuffs, 12, 14 preferably have a soft, removable cover (not shown), which may be a paper based or bubble-wrap type padding to cover the respective inner surfaces of the cuffs 12, 14. Preferably, the cuff padding is disposable. The cuff padding improves cleanliness of the apparatus as well as the patient's comfort. When used in the home, a soft, washable fabric covering preferably covers the cuffs 12, 14.

Referring to FIG. 2, the base cuff 12 includes a mounting base 38 secured to the upper portion of the cuff 12. The mounting base 38 may be integral to the cuff 12, or may be rigidly attached by hardware. The mounting base 38 is preferably threaded to allow mating with a coupling 50. The coupling 50 also receives spacer 52, which has at the end opposite the base cuff 12 a generally flat portion 58. The generally flat portion 58 of the spacer 52 acts as a stop when assembled with the upper cuff 14 assembly, as described below.

Referring to FIG. 3, a hollow base tube 48 is positioned onto the top of the base cuff 12. The base tube 48 is sized such that its inner diameter forms a snug fit with the outside diameter of the coupling 50. Preferably, the hollow base tube 48 is secured to the coupling 50 via hardware 30. A compression clamp 46 mates with the hollow base tube 48. In a preferred embodiment, the compression clamp 46 includes a handle 70 for tightening the clamp. It is noted that many forms of compression clamps may be used to accomplish the same purpose.

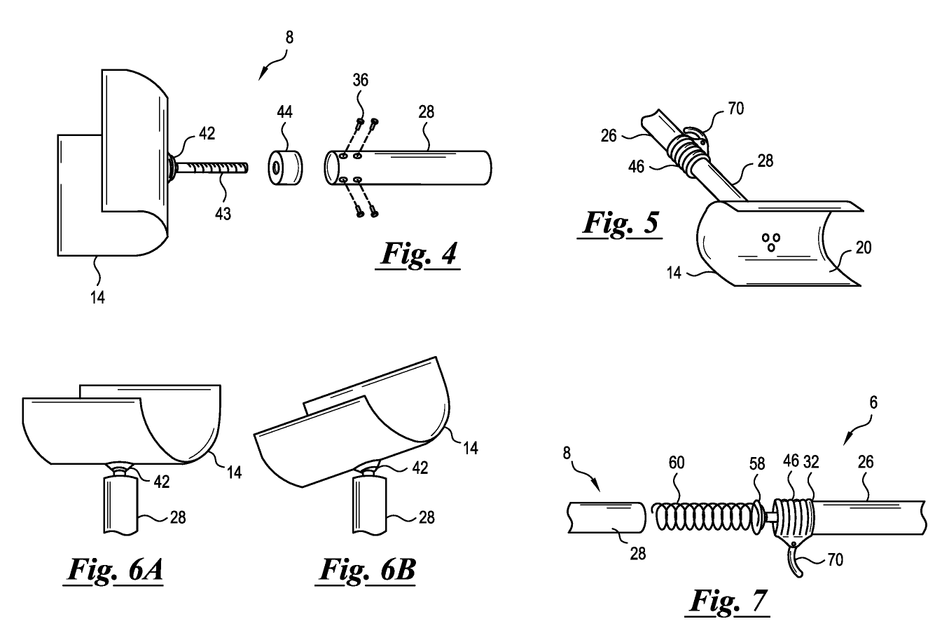

Referring to FIG. 4, the upper cuff 14 includes a mounting base 42 secured to the outer portion go the upper cuff 14. The mounting base 42 may be integral to the cuff 12, or may be rigidly attached by hardware. The mounting base 42 preferably includes a threaded rod 43 to allow mating with a second coupling 44. A hollow upper tube 28 is positioned onto the coupling 44. The upper tube 28 is sized such that its inner diameter forms a snug fit with the outside diameter of the second coupling 44. Preferably, the hollow upper tube 28 is secured to the coupling 440 via hardware 36.

The mounting base 42 of the upper cuff 14 preferably comprises an articulating joint, such a ball type joint. This allows the upper cuff 14 to pivot in all directions thereby allowing the upper cuff 14 to conform to a patient's unique body type, as shown in FIGS. 6A and 6B.

Referring to FIG. 7, the base cuff assembly 6 and the upper cuff assembly 8 are assembled by sliding the upper tube 28 into the base tube 26. It is noted that the outer diameter of the upper tube is sized slightly smaller than the inner diameter of the base tube 26 to provide a snug, while also allowing the components to easily slide. A compression spring 60 is placed inside of the upper tube 28 and rests on the stop 58 of the base assembly 6. The compression clamp 46 is then tightened by way of handle 70. The spring 60 ensures a threshold amount of force is required in order to move the upper cuff assembly 8 towards the base cuff assembly 6. In use, a patient's leg may be positioned in the upper cuff 14 while a caregiver needs to adjust the apparatus 10. In this scenario, the compression spring 60 provides a bias to the weight of the patient's leg, enabling the caregiver to adjust the height of the apparatus 10 easily.

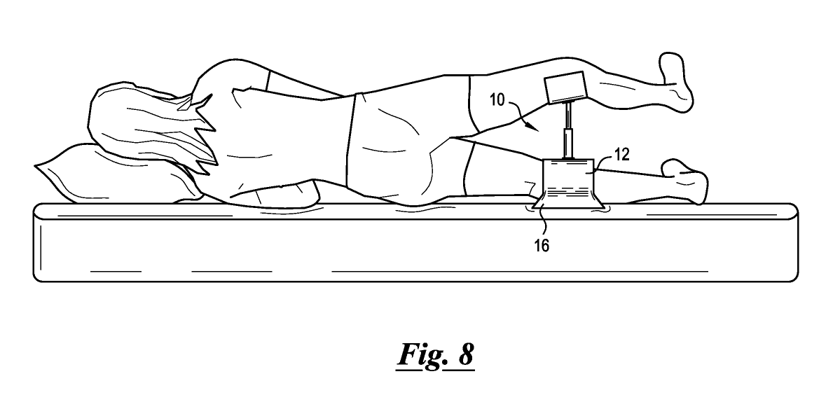

The apparatus 10 is preferably used in a manner consistent with FIG. 8. Preferably, a patient is positioned on their side. The apparatus 10 is inserted between the patient's legs, approximately at the knee. The exact positioning of the device will depend on the patient's body type, comfort, and desired degree of abduction. As can be seen in FIG. 8, the articulating joint 42 of the upper cuff 14 allows the apparatus 10 to conform to various body types and improve patient comfort. The flange 16 of the base cuff 12 can also be seen resting on the bed, thereby improving stability of the apparatus 10.

Preferably, the compression clamp 46 provides compression, and therefore locks the upper tube 28 in place, in a default position. In this manner, a caregiver must actuate the compression clamp 46 in order to adjust the distance between the cuffs 12, 14. A mechanical stop may also be provided such that the upper tube 28 is not forced out of the base tube 26 by the compression spring 60. However, this scenario would be eliminated by using a compression clamp that has a compressed default (not actuated) position.

The material of construction of the apparatus 10 may be any suitable material that is easily cleaned. Stainless steel, anodized aluminum, and various thermoplastics are lightweight and easily cleaned and steam sterilized, and therefore are suitable materials for construction.

In an alternative embodiment of the present invention, referring to FIG. 9, the telescoping mechanism between the base cuff and the upper cuff as described in the embodiment above is replaced by a fixed length support arm 102. The base cuff 104 again includes a mounting base 106, and again preferably includes a flange, as described above with reference to FIGS. 1 through 3, to provide a stable base for freestanding use. The upper cuff 108 also includes a mounting base 110. In this embodiment, the mounting bases 106, 110 are rigid mounting based that do not articulate or swivel. The support arm 102 couples with each mounting base 106, 110 in order to assemble the apparatus 100. The manner in which the support arm 102 interconnects with each mounting base 106, 100 is preferably a snap connection, but may be any suitable removable connection.

Alternatively, referring to FIG. 10, the upper mounting base 112 includes an articulating joint, such as a ball type joint as described above with reference to FIGS. 4 and 6. In this embodiment, the apparatus 120 provides patient comfort by way of an upper cuff 108 that articulates to conform to the patient, while also provided a low cost alternative by way of a fixed length support arm 102. Preferably, in the embodiments described with reference to FIGS. 9 and 10, the support arm 102 is made from a low cost material, such as a thermoplastic, and is disposable. These embodiments are ideal for in-home use, and provide a low cost alternative to the more complex preferred embodiment described above with reference to FIGS. 1 through 7. Still referring to FIG. 10, support arms 114, 116 of varying lengths may be interchanged with support arm 102 in order to achieve varying degrees of abduction. In this manner, a low cost, disposable apparatus for leg abduction is realized.

Having thus described in detail a preferred selection of embodiments of the present invention, it is appreciated and will be apparent to those skilled in the art that many physical changes could be made in the apparatus without altering the inventive concepts and principles embodied therein. The present embodiments are therefore to be considered in all respects as illustrative and not restrictive, the scope of the invention being indicated by the appended claims rather than by the foregoing description, and all changes which come within the meaning and range of equivalency of the claims are therefore to be embraced therein.

What is claimed is:

1. An apparatus for abducting the legs of a patient, the apparatus comprising:
first and second cuffs, wherein the cuffs are adapted for securing the patient's legs, and the first cuff comprises a flange adapted for stabilized the apparatus on a flat surface;
a telescoping support arm having a first and second end, wherein the first end of the support arm is coupled to the second cuff, and the support arm is adjustable in a first dimension; and
a pivotable joint coupled between the second cuff and the second end of the support arm adapted for allowing motion of the second cuff.

2. The apparatus of claim 1, wherein the telescoping support arm further comprises:
a first hollow tube coupled to the first cuff;
a second hollow tube coupled to the pivotable joint; and
a compression clamp adapted to secure to the first and second hollow tube in a fixed position.

3. The apparatus of claim 2, wherein the telescoping support arm further comprises:
a compression spring adapted to bias the first hollow tube and the second hollow tube.

4. The apparatus of claim 1, wherein the pivotable joint pivots in multiple planes.

5. The apparatus of claim 1, wherein the cuffs are substantially C shaped.

6. The apparatus of claim 1, wherein the interior surface of the cuffs are covered by a soft, washable padding.

7. The apparatus of claim 1, wherein the apparatus extends substantially perpendicular to a plane formed by the individual's legs.

8. An apparatus for abducting the legs of a patient, the apparatus comprising:
first and second cuffs, wherein the cuffs are adapted for securing the patient's legs, and the first cuff comprises a flange adapted for stabilized the apparatus on a flat surface;
a fixed length support arm having a first and second end, wherein the first end of the support arm is removably coupled to the second cuff; and
a pivotable joint coupled between the second cuff and removably coupled to the second end of the support arm, wherein the pivotable joint is adapted for allowing motion of the second cuff.

9. The apparatus of claim 8, wherein the pivotable joint pivots in multiple planes.

10. The apparatus of claim 8, wherein the cuffs are substantially C shaped.

11. The apparatus of claim 8, wherein the interior surface of the cuffs are covered by a soft, washable padding.

12. The apparatus of claim 8, wherein the apparatus extends substantially perpendicular to a plane formed by the individual's legs.

13. The apparatus of claim 8, wherein the fixed length support is adapted to be replaced with other fixed length supports of varying length to accommodate patients of different sizes.
